(19) United States
(12) Patent Application Publication
(10) Pub. No.: US 2004/0253139 A1
(43) Pub. Date: Dec. 16, 2004

(54) Environmental surface disinfectant

(76) Inventor: Robert Michael Denton, Delray Beach, FL (US)

Correspondence Address: STOCKWELL & ASSOCIATES, PSC, 861 CORPORATE DRIVE, SUITE 201, LEXINGTON, KY, 40503, US

(21) Appl. No.: 10/461,003

(22) Filed: Jun. 14, 2003

(51) Int. Cl. A61K 31/21

(52) U.S. Cl. 422/28; 514/546

(57) ABSTRACT

An environmental surface disinfectant and its method of use are disclosed. The inventive environmental surface disinfectant comprises an effective concentration of an alkyl ester of lactic acid. Ethyl lactate is a preferred alkyl ester of lactic acid. Disinfection of an environmental surface is achieved by applying the disinfectant of the invention to the surface.

### CROSS-REFERENCE TO RELATED APPLICATIONS

This application claims priority from provisional application Ser. No. 60/388,606 filed Jun. 13, 2002.

### FIELD OF THE INVENTION

This invention relates to a composition and method of using compositions of alkyl esters of lactic acid as non-toxic, environmentally friendly disinfectants of solid substrates. The disinfectants are usable in a variety of contexts without corroding or chemically attacking the material in such surfaces.

### BACKGROUND OF THE INVENTION

Environmental surface disinfectants are useful for removing pathogens on inanimate surfaces in a variety of settings. In household settings, environmental surface disinfectants are useful to prevent the transmission of pathogens from surfaces in the kitchen and bathroom, among others. In medical settings, environmental surface disinfectants are useful for removing pathogens from reusable patient-care equipment and other surfaces contacted by patients. In recreational settings, environmental surface disinfectants are useful to prevent the transmission of pathogens by recreational equipment, such as scuba gear.

A variety of environmental surface disinfectants are known in the art. Triclosan is perhaps the most common environmental surface disinfectant for household use. However, there is public concern regarding its chemical similarity to dioxins, which are known to harmful to human health.

Alcohols, such as isopropyl ethyl alcohol, are also widely used as environmental surface disinfectants. Although alcohols are well known in the art for their wide germicidal activity, alcohols evaporate rapidly, making extended contact times difficult to achieve. This factor precludes the practical use of alcohols as large surface disinfectants. Another disadvantage is that alcohols must be used at very high concentrations, usually between 70-95% concentration, for effectiveness.

Halogens, such as iodine or chlorine compounds, are also well known in the art. Although the halogens provide wide germicidal activity, they have several undesirable properties. Halogen containing compositions are generally corrosive. Iodine containing compositions may also stain the surface being disinfected. Chlorine containing compositions are generally toxic.

Quaternary ammonium compounds are also well known in the art. However, the quaternary ammonium compounds have a limited germicidal range and have limited effectiveness in soaps, detergents and hard water salts.

Phenolic compounds are well known in the art. However, phenolic compounds have an unpleasant odor, and leave a sticky residue on surfaces.

Coal tar distillates, such as Cresol and Cresylic Acid, are well known in the art. However, coal tar distillates have several undesirable qualities as environmental surface disinfectants. Coal tar distillates are corrosive and toxic at high concentrations. In addition, coal tar distillates emit noxious gases.

Aldehydes, such as glutaraldehyde, are also well known in the art. However, aldehydes suffer the disadvantage of being moderately toxic.

Oxidizing agents, such as hydrogen peroxide and potassium permanganate, are also well known in the art. However, oxidizing agents have undesirable qualities. Oxidizing agents are corrosive and exposure to high concentrations (5% or greater) can result in eye and skin irritation.

There is thus a need in the art for an environmental surface disinfectant which may efficiently be employed for eliminating microorganisms, but which at the same time is non-toxic, has a mild odor, is compatible with cleaning compounds and does not react with solid substrates.

### SUMMARY OF THE INVENTION

A composition and a method for disinfecting an environmental surface using that composition is provided. The surface is contacted with a biocidal effective amount of a composition comprising alkyl esters of lactic acid; and optionally, other common elements present in surface cleaners, such as water, surfactants, and odorants among others.

### DETAILED DESCRIPTION OF THE INVENTION

Detailed descriptions of the preferred embodiments are provided herein. It is to be understood, however, that the present invention may be embodied in various forms. Therefore, specific details disclosed herein are not to be interpreted as limiting, but rather as a basis for the claims and as a representative basis for teaching one skilled in the art to employ the present invention in virtually any appropriately detailed system, structure or manner.

The present invention involves using alkyl esters of lactic acid, which have heretofore been unappreciated for their surprising disinfectant properties, to disinfect environmental surfaces.

In accordance with the present invention, it has surprisingly been discovered that compositions based on alkyl lactate esters have remarkable antibacterial properties. The compositions of the invention comprise alkyl lactates wherein the alkyl group has 1 to 12 carbon atoms.

According to a first embodiment of the present invention, the environmental surface disinfectant contains a lactate as the antimicrobial ingredient. This lactate can be either methyl lactate, butyl lactate, or propyl lactate.

According to another embodiment of the present invention, the environmental surface disinfectant contains ethyl lactate as the antimicrobial ingredient. Ethyl lactate is non-toxic, and is used in foods and cosmetics. In addition, ethyl lactate has a mild odor and is not corrosive or reactive with most solid substrates.

Antimicrobial activity of the composition is attained at concentrations of ethyl lactate greater than two per cent, achieving maximal antibacterial activity at ethyl lactate concentrations of approximately fifty percent. Accordingly, the preferred concentration range for ethyl lactate when employed in the antimicrobial compositions is between two and fifty percent, although the range can be as high as one hundred percent. The composition of the invention may contain one or several other compounds with antiseptic properties, perfumes or other customary additives and auxiliaries such as surfactants. The compositions according to the invention can be prepared by mixing the individual components together successively, if necessary with heating. No particular order need be adhered to during this process.

### EXAMPLE 1

The following example demonstrates that ethyl lactate is a potent bactericide. Using a modification of AOAC Method 956.17, the lower effective concentration for ethyl lactate was tested against E. coli (ATCC 25922) at three reaction times at varying concentrations of ethyl lactate. As the results in Table 1 show, exposure of E. coli to a 15% solution of ethyl lactate for 10 minutes eliminated bacterial growth.

TABLE 1
Lower effective concentration of ethyl lactate against
E. coli (ATCC 25922) at final concentration of CA.
100 billion colonies for 24 hours.

| | Ethyl lactate, | Time | | |
|---|---|---|---|---|
| Label | concentration^{a} | 5 min. | 10 min. | 15 min. |
| S-1^{b} | 10% | +^{c} | + | + |
| S-2 | 50% | −^{d} | − | − |
| S-3 | 20% | − | − | − |
| S-4 | 30% | − | − | − |
| S-5 | 40% | − | − | − |
| S-6 | 15% | + | − | − |
| S-7 | 20% | − | − | − |

^{a}Diluted with water, plus challenge bacteria (0.5 ml of 1 trillion E. Coli added to 5.0 ml of each sample).
^{b}S = Sample
^{c}+ = Bacterial growth observed
^{d}− = No Bacterial growth observed

### EXAMPLE 2

The following example shows one preferred embodiment according to the invention. A formulation is prepared as follows:

| Ingredient | Weight % |
|---|---|
| Ethyl Lactate | 16.0% |
| Fragrance | 1.0% |
| Water | 83.0%. |

Fragrance is added first to the ethyl lactate and then mixed in the water.

Turning to Table 2, there is shown that the bactericidal efficiency of the formulation in Example 2 is comparable to that of 10% Clorox at the challenge times observed.

TABLE 2
Comparison of preferred embodiment with diluted Clorox
against E. Coli (ATCC 25922) at concentration of CA.
100 billion colonies for 24 hours.

| | | Time | | |
|---|---|---|---|---|
| Label | concentration^{a} | 5 min. | 10 min. | 15 min. |
| Clorox | 10% | N^{b} | N | N |
| Formulation | 100% | N | N | N |

^{a}Diluted with water, plus challenge bacteria (0.5 ml of 1 trillion E. Coli added to 5.0 ml of each sample).
^{b}N = No Bacterial growth observed

Turning to Table 3 there is shown with a conventional challenge test that the formulation eliminates E. Coli with a very high efficiency.

TABLE 3
Results of challenge test using E. Coli (ATCC 25922).

| Challenge volume: | 5.0 ml |
|---|---|
| Product volume: | 0.5 ml |
| E. Coli level of challenge liquid: | 100 Billion CFU/ml* |
| E. Coli level of treated liquid: | <1 CFU/ml |
| E. Coli removal efficiency: | >99.999% |

*CFU/ml = Colony forming unit per milliliter.

Tables 4 and 5 show with a conventional challenge test that the formulation eliminates the fungi Penicillum pinopillum and Aspergillus niger with a very high efficiency.

TABLE 4
Results of challenge test using Penicillum pinopillum.

| Challenge volume: | 5.0 ml |
|---|---|
| Product volume: | 0.5 ml |
| P. pinopillum level of challenge liquid: | 0.4 Million CFU/ml* |
| P. pinopillum level of treated liquid: | 300 CFU/ml |
| P. pinopillum removal efficiency: | >99.25% |

*CFU/ml = Colony forming unit per milliliter.

TABLE 5
Results of challenge test using Aspergillus niger.

| Challenge volume: | 5.0 ml |
|---|---|
| Product volume: | 0.5 ml |
| A. niger level of challenge liquid: | 0.9 Billion CFU/ml* |
| A. niger level of treated liquid: | <1200 CFU/ml |
| A. niger removal efficiency: | >98.5% |

*CFU/ml = Colony forming unit per milliliter.

It is thus apparent from the results of the Examples described above that the method of using alkyl lactates as environmental surface disinfectants according to the invention is a valuable method of disinfecting, possessing surprisingly good antimicrobial effects. The environmental surface disinfectant according to the present invention is particularly well-suited in medical and food preparation contexts also. The disinfectant according to the invention is moreover easy to produce by a simple mixing process.

One skilled in the art will appreciate that the particular alkyl lactate chosen can provide a more immediate and more effective reduction in microbial count depending on the microflora targeted. Accordingly, the described examples are merely exemplary and are in no way limiting.

The concentration of alkyl lactate in accordance with the present invention should be sufficient to effect the desired reduction in bacterial count over a reasonable time frame. One skilled in the art will recognize that concentration will depend upon a variety of factors, including the particular alkyl lactate employed, the targeted bacterial microflora, and the nature of the other compounds in the bacterial microflora may require prolonged treatment involving multiple applications of compositions of the present invention.

Suitable concentrations of alkyl lactate can be determined by conventional range-finding techniques known to those of ordinary skill in the art. Several standard methods for determining the antimicrobial efficacy of various concentrations of alkyl lactates as on the various resident and transient microflora of environmental surfaces are well known to those of ordinary skill in the art.

Seen as a whole, therefore, the compositions according to the invention are especially suitable as environmental surface disinfectants.

The invention has been described in detail with particular reference to preferred embodiments thereof, but it will be understood that variations and modifications can be effected within the spirit and scope of the invention.

What is claimed is:

1. [Cancelled]

2. [Cancelled]

3. A surface disinfectant comprising methyl lactate and a composition selected from the group comprising water, odorants, surfactants, disinfectants, detergents, or a combination thereof.

4. [Cancelled]

5. A surface disinfectant comprising butyl lactate and a composition selected from the group comprising water, odorants, surfactants, disinfectants, detergents, or a combination thereof.

6. [Cancelled]

7. A surface disinfectant comprising propyl lactate and a composition selected from the group comprising water, odorants, surfactants, disinfectants, detergents, or a combination thereof.

8. [Cancelled]

9. A surface disinfectant comprising ethyl lactate and a composition selected from the group comprising water, odorants, surfactants, disinfectants, detergents, or a combination thereof.

10. The surface disinfectant of claim 9 wherein the concentration of ethyl lactate is between 2% and 100% by volume.

11. The surface disinfectant of claim 9 wherein the concentration of ethyl lactate is between 20% and 50% by volume.

12. A method of disinfecting a surface, comprising:
applying a disinfectant to a surface, wherein said disinfectant contains an effective amount of at least one alkyl ester of lactic acid, wherein the alkyl group of said alkyl ester has 1 to 12 carbon atoms.

13. The method of claim 12, wherein said alkyl ester of lactic acid is ethyl lactate.

14. The method of claim 13, wherein said disinfectant also contains a composition selected from the group comprising water, odorants, surfactants, disinfectants, detergents, or a combination thereof.

15. [Cancelled]

16. [Cancelled]

17. [Cancelled]

18. [Cancelled]
